(19) United States
(12) Patent Application Publication
(10) Pub. No.: US 2010/0239589 A1
(43) Pub. Date: Sep. 23, 2010

(54) Methods and Compositions for Ameliorating Diabetes and Symptoms Thereof

(75) Inventors: Niels-Bjarne Woods, San Diego, CA (US); Maziyar Saberi, Millbrae, CA (US); Jerrold M. Olefsky, San Diego, CA (US); Inder M. Verma, San Diego, CA (US); Carl de Luca, San Diego, CA (US)

Correspondence Address: CATALYST LAW GROUP, APC, 9710 SCRANTON ROAD, SUITE 280, SAN DIEGO, CA, 92121, US

(73) Assignee: Salk Institute for Biological Studies, La Jolla, CA (US)

(21) Appl. No.: 12/711,073

(22) Filed: Feb. 23, 2010

Related U.S. Application Data
(60) Provisional application No. 61/154,725, filed on Feb. 23, 2009.

(51) Int. Cl. A61K 39/395; A61P 3/00; A61P 3/10; A61P 3/04; A61P 9/00; C12Q 1/68

(52) U.S. Cl. 424/143.1; 435/6

(57) ABSTRACT

The present invention relates generally to the Tlr4 signaling pathway specifically in the hematopoietic system and its contribution to insulin resistance of liver and adipose tissue. The hematopoietic component expressing Tlr4 is a principle propagator of immune signaling and results in insulin resistance. Furthermore, disclosed herein are methods and compositions for treating or preventing disorders associated with insulin resistance using a Tlr4 antagonist.

### RELATED APPLICATIONS

The present application is related to, and claims priority from, U.S. Provisional Patent Application No. 61/154,725, filed Feb. 23, 2009, the entire disclosure of which is herein incorporated by reference.

### GOVERNMENT SUPPORT

This invention was made with government support under Grant No. DK033651, DK0748468, and T32 DK007494 awarded by the National Institutes of Health. The Government has certain rights in the invention

### FIELD OF INVENTION

The present invention relates generally to immunology, specifically the TLr4 signaling pathway. More particularly, it concerns methods and compositions relating to the suppression of TLr4 signaling in relation to disorders associated with insulin resistance.

### BACKGROUND OF THE INVENTION

The immune system responds innately to invading pathogens. Members of the toll-like receptor (Tlr) gene family convey signals stimulated by these factors, activating signal transduction pathways that result in transcriptional regulation thereby stimulating immune function. Tlr's play a critical role in activating the innate immune response, and consequently, have been implicated in the induction of insulin resistance in obesity. Chronic low-grade tissue inflammation has recently garnered considerable attention as a necessary contributor to insulin resistance in obesity. Recent evidence shows that chronic inflammation is a central contributing factor in the development of insulin resistance in obesity, the pathway(s) that transduce the inflammatory signal in obesity are unclear.

Insulin resistance is a major metabolic defect in obesity, and is associated with increased risk of various diseases, such as type 2 diabetes, hypertension arid coronary heart disease 1. “Type II Diabetes” or “non-insulin dependent diabetes mellitus” (NIDDM) is the form of diabetes which is due to a profound resistance to insulin stimulating or regulatory effect on glucose and lipid metabolism in the main insulin-sensitive tissues, muscle, liver and adipose tissue. This resistance to insulin responsiveness results in insufficient insulin activation of glucose uptake, oxidation and storage in muscle and inadequate insulin repression of lipolysis in adipose tissue and of glucose production and secretion in liver. When these cells become desensitized to insulin, the body tries to compensate by producing abnormally high levels of insulin and hyperinsulemia results. Hyperinsulemia is associated with hypertension and elevated body weight. Since insulin is involved in promoting the cellular uptake of glucose, amino acids and triglycerides from the blood by insulin sensitive cells, insulin insensitivity can result in elevated levels of triglycerides and LDL which are risk factors in cardiovascular diseases.

In recent years, chronic, low-grade inflammation has emerged as an important contributor to the etiology of insulin resistance in obesity, and because the expansion of adipose tissue mass is an obvious corollary of obesity, much research has focused on adipose tissue as a potential site of this inflammation. Indeed, obese adipose tissue is characterized by increased expression of inflammatory genes, such as tumor necrosis factor (TN F)-alpha, interleukin (IL)-6, Regulated upon Activation Normal T-cell Expressed and Secreted (RANTES), and monocyte chemoattractant protein (MCP)-1, as well as increased infiltration by immune cells, particularly macrophages 2-4.

Macrophages are an important modulator of inflammation, through their capacity to secrete a variety of proinflammatory chemokines and cytokines. In fact, adipose tissue macrophages (ATMs) appear to be responsible for much of the increase in inflammation in adipose tissue with obesity 2,3.

Consistent with a role for macrophages and inflammation in the pathogenesis of insulin resistance, the deletion of the two primary inflammatory pathways in macrophages, namely the inhibitor of I_{K}B kinase/nuclear factor _{K}B (IKK1NF_{K}B), 5, and c-Jun NH₂ terminal kinase 1/activator protein (JNK1/AP1), 6, pathways attenuates obesity-induced insulin resistance. Thus, preventing the propagation of inflammatory signals within macrophages is sufficient to mitigate obesity-induced insulin resistance. Nonetheless, the upstream components or pathways that detect, initiate and activate the proinflammatory IKK1NF_{K}B and JNK1AP1 pathways remain to be fully elucidated.

Potential “sensors” that may link obesity to inflammation are the toll-like family of receptors (Tlr's); the pattern recognition receptors that play critical roles in innate immunity 7,8. Relevant to obesity and inflammation, Tlr's, particularly Tlr2 and Tlr4, are highly expressed in macrophages and adipose tissue. Tlr4 is an attractive candidate for linking innate immunity to insulin resistance, because it is expressed in most cell types, and Tlr4 is also a receptor for fatty acids, which are increased in obesity 9,10.

Fatty acids (particularly saturated fatty acids) can activate Tlr2/4 resulting in activation of the IKK1NF_{K}B and JNK1 pathways, with enhanced secretion of pro-inflammatory chemokines and cytokines (e.g. TNFα) 9,10. In contrast, in vitro fatty acid-induced activation of JNK and IKK, or induction of proinflammatory cytokine expression or secretion, is prevented by siRNA-mediated knockdown of Tlr2/4 in the RAW264.7 macrophage cell line, or in macrophages from Tlr4 knockout mice 4,11.

Since obesity is characterized by elevated fatty acid levels and flux 12,13, the fact that fatty acids can stimulate a receptor that, in turn, activates inflammatory pathways provides a potentially important link between obesity, inflammation and insulin resistance. In support of this, Tlr4 expression is increased in adipose tissue in obesity, and in proinflammatory macrophages 4,11. In addition, obesity due to high-fat diet (HFD) feeding and insulin resistance caused by a lipid-plus-heparin infusion were attenuated in Tlr4 knockout mice, in parallel with decreased inflammation in both liver and adipose tissue 11,14. However, while these studies clearly implicate Tlr4 in the development of lipid and obesity-induced insulin resistance, the specific tissue(s) in which Tlr4 depletion works to protect mice from insulin resistance remains to be defined.

While it has been known that innate immunity/inflammation is involved in promoting insulin resistance, the precise link between the pro-inflammatory signaling from Trl4, and insulin resistance has not yet been discovered. Specifically, it has not been determined what signaling pathway downstream of the Tlr4 pathway was involved in blocking insulin function, nor whether the Tlr4 receptor was acting on the liver, adipose, muscle cells themselves or on the inflammatory cells (hematopoietic derived cells). This invention provides the hematopoietic component expressing Tlr4 is a principle propagator of immune signaling and results in insulin resistance.

### SUMMARY OF THE INVENTION

This invention is based, at least in part, on the discovery that knockout of Tlr4 signaling in macrophages reverses insulin resistance in adipose tissue and liver in HFD fed, obese mice. This protection occurs in parallel with a marked reduction in macrophage infiltration in adipose tissue, and reduced inflammatory markers in adipose and liver. Altogether, these data indicate the importance of innate immunity and hematopoietic derived cells, particularly macrophages and Kupffer cells, in the induction of insulin resistance in obesity.

Another embodiment, of the present invention also identifies Tlr4 in hematopoietic derived cells as a potential target for the therapeutic treatment of insulin resistance.

Another aspect of the invention, is a method wherein a sample cell expressing Tlr4 is contacted with a test compound that inhibits Tlr4 expression in the cell. A test compound that inhibits Tlr4 expression and/or activity is a Tlr4 antagonist and therefore a therapeutic compound for the treatment of insulin resistant diseaies.

Another aspect of this invention is to identify compounds ie., test compounds, agents, or antagonists (e.g. proteins, peptides, peptidomimetics, peptoids, small molecules or other drugs) that modulate the expression of T1r4.

Another aspect of the present invention is to identify compounds ie., test compounds, agents, or antagonists (e.g. proteins, peptides, peptidomimetics, peptoids, small molecules or other drugs) that modulate the activity of Tlr4.

In still another aspect of the invention laboratory diagnostic tests can formulated for detecting the presence and or level of Tlr4 expression in specimens, for determining a treatment regimen.

In another aspect the invention includes methods for treating a disorder associated with insulin resistance in a subject by administering to the subject a therapeutically effective amount of a composition including an antagonist of Toll-like receptor 4.

In another aspect the invention includes methods for preventing a disorder associated with insulin resistance in a subject by administering to the subject a therapeutically effective amount of a composition including an antagonist of Toll-like receptor 4.

In still another aspect of the invention is a pharmaceutical composition comprising a Tlr4 antagonist described herein and a physiologically acceptable carrier.

Therefore, another aspect of the present invention is a method to treat or prevent type 2 diabetes in patients by using small molecule inhibitors of Tlr4 receptor binding to its ligand or inhibitors of Tlr4 signaling.

Thus in accordance with the present invention is a method of ameliorating diabetes.

It is contemplated that any method or composition described herein can be implemented with respect to any other method or composition described herein.

Unless otherwise defined, all technical and scientific terms used herein have the same meaning as commonly understood by one of ordinary skill in the art to which this invention belongs. Methods and materials are described herein for use of the present invention; other suitable methods and materials known in the art can also be used. The materials and methods, and examples are illustrative only and not intended to be limiting. All publications, patent applications, patents and other references mentioned herein, are incorporated by reference in their entirety. In case of conflict, the present specification, including definitions will control.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 shows Hematopoietic homeostasis is maintained in mice following bone marrow transplantation of Tlr4 deleted hematopoietic cells. FIG. 1A shows real-time PCR analysis for the wt Tlr4 genomic locus in peripheral blood mononuclear cells from transplanted mice, demonstrating near complete reconstitution of white blood cells in bone marrow transplant recipient mice with Tlr4 deletion mutant hematopoietic cells (BMT-Tlr4^{-/-})(n=8). FIG. 1B and FIG. 1C, show hematogram analyses demonstrating normal blood cell lineage distributions including monocytes, lymphocytes and neutrophils, in mice BMT-Tlr4^{-/-}), as compared with control mice transplanted with wild type Tlr4 (BMT-wt) cells. High fat diet fed mice had slightly altered lineage distributions with slightly increased neutrophil counts and decreased lymphocyte counts, but this was similar for both the BMT-wt and BMT-Tlr4^{-/-} mice. FIG. 1D and FIG. 1E show weight gain of mice given high fat diet (HFD) or normal chow diet (NCD) for 12 weeks following BMT, with no statistical difference in weight gain or food intake/day between BMT-Tlr4^{-/-} and BMT-wt mice. FIG. 1F and FIG. 1G shows cross section and coronal section views using 3D magnetic resonance imaging (MRI) of mice and software based analyses of tissue volumes to determine the total weight of visceral adipose tissues and nonfat tissues demonstrating similar distribution of adipose tissue in the BMT-wt and BMT-Tlr4^{-/-} mice. Red arrows signify visceral fat, and yellow dots signify hepatic tissue. FIG. 1H shows weight gain results of a control experiment where the global Tlr4 knockout mice and wild type mice were placed on HFD. No weight gain was seen in the global Tlr4 knockout mice on a HFD. Statistical analyses were performed using the T-test.

FIG. 2 shows Insulin tolerance tests in BMT-Tlr4^{-/-} mice on HFD show normal insulin sensitivity. The insulin tolerance test (ITT) performed on bone marrow transplanted mice reveals a significantly increased insulin sensitivity in BMT-Tlr4-/mice fed a high fat diet (HFD) compared to BMT-wt mice also fed HFD, FIG. 2A. The glucose tolerance test (GTT) reveals less efficient glucose clearing over time in obese mice compared to normal weight control mice for either BMT group, Panel B. * and ** signifies statistical significance between BMT-Tlr4^{-/-} and BMT-wt glucose levels for p<0.05 and p<0.01, respectively, using t-test. FIG. 2C and FIG. 2D reveal the partial correction of insulin levels in obese BMT-Tlr4^{-/-}, mice compared to BMT-wt mice. Area-under-curve analysis of insulin data from FIG. 2C shows a statistical difference between BMT-Tlr4^{-/-}, and BMT-wt mice both fed HFD.

FIG. 3 illustrates the hyperinsulinemic euglycemic clamp test showing obese BMT-Tlr4^{-/-} mice have normal insulin sensitivity of hepatocytes and adipocytes. FIG. 3A shows glucose infusion rates during clamp test of mice with the BMT-Tlr4^{-/-} mice showing partial correction of insulin function as measured by partially restored glucose infusion rates (n=8). FIG. 3B shows the hepatic glucose production (HGP) both at basal levels and during the clamp test. On HFD, HGP in the BMT-Tlr4^{-/-} mice was not different from HGP on chow diet, whereas, HFD led to an increase in HFP in the BMT-wt group. The percent suppression of HGP is shown in FIG. 3C. FIG. 3D demonstrates the percentage suppression of free fatty acid secretion from adipose tissue, with obese BMT-Tlr4^{-/-} mice showing normalized adipocyte insulin sensitivity. FIG. 3E shows the insulin stimulated glucose disposal rate (IS-GDR) on NCD or HFD for the two genotypes. * signifies statistical significance between mice groups p<0.05, using t-test, n>8.

FIG. 4 shows insulin sensitivity in hepatocytes of obese mice correlates with reduced inflammatory signaling of Tlr4^{-/-} hepatic macrophages. CD11b liver macrophages (Kupffer cells) play an important role in regulating inflammation in the liver. FIG. 4A and FIG. 4B show the increase in liver weight of HFD fed mice compared to NCD mice, and the near complete reconstitution of Tlr4-1 Kupffer cells in the liver of obese mice following bone marrow transplantation. Homogenized liver tissue from bone marrow transplanted mice was FACS sorted for the macrophage specific marker CD11b and real time PCR performed to assay for wild type Tlr4 genomic DNA content. FIG. 4C shows the fold reductions of macrophage specific and non-specific pro-inflammatory cytokines, chemoaUractants, and signaling molecules of BMT-Tlr4^{-/-} mice compared to BMT-wt mice fed a HFD. FIG. 4D and FIG. 4E confirm levels of TNF-α and the chemoaUractant RANTES via ELISA. Western blot for JNK1/2 demonstrates that BMT-Tlr4^{-/-} mice on a HFD have decreased JNK1/2 signaling compared to BMT-wt mice (FIG. 4F).

FIG. 5 illustrates the Reduced inflammatory cell recruitment to adipose tissue in 8MTTlr4−/− mice on HFD. FIG. 5A shows decreased expression of common proinflammatory cytokines in adipose tissue of m BMT-Tlr4^{-/-} mice compared to BMT-wt mice fed a HFD. FIG. 5B-FIG. 5E show histological analysis of adipose tissue stained with MAC2 antibody for macrophage detection. Significantly increased macrophage infiltration between adipocytes is detectable and visually quantitated as crown-like-structures with significant reductions in BMT-Tlr4^{-/-} mice compared to BMT-wt mice fed HFD (Panel F). * and ** signifies statistical significance between and BMT-Tlr4^{-/-} BMT-wt levels for p<0.05 and p<0.01, respectively, using T-test.

FIG. 6 shows Lentiviral vector mediated knockdown of Tlr4 in a hematopoietic stem cell gene therapy setting maintains insulin sensitivity in HFD mice. Lentiviral vector driving expression of small interfering RNA (siRNA) targeted against Tlr4 in bone marrow transplanted mice of transduced and sorted hematopoietic stem cells, yields partial but significant knockdown of Tlr4 expression in peripheral blood mononuclear cells of transplanted mice as determined by real time PCR, (n=3), FIG. 6A. FIG. 6B shows average weights of mice fed either a high fat diet or a normal chow diet. Insulin tolerance test (ITT) was performed at 8 weeks posUransplant with LV-siTlr4 and control vector transduced bone marrow mice receiving normal chow (FIG. 6C) or high fat diet (FIG. 6D).

FIG. 7 shows NFₖB Luciferase activity in THP-1 cells (Human Acute

Moncytic Leukemia Cell Line). THP-1 cells were transduced with a lentiviral 5×-NFkB-luciferase-mPGK-mcherry construct and sorted for mcherry. In conditions with 10 uM IKK2 inhibitor or 0.1, 1, or 5 ug antagonist (CRX-526, Glaxo Smith Kline), cells were pre-treated for 1 hr. Cells were then treated with 10 ng/ml LPS (Sigma) or 10 ng/ml human TNFα (Sigma) for 6 hrs. After 6 hr LPS or TNFα stimulation, cells were harvested and analyzed for luciferase activity using Steady Glo Reagent (Promega), 23.

FIG. 8 illustrates THP-1 cells that were transduced with a lentiviral 5×-NFkB-luciferase-mPGK-mcherry construct¹ and sorted for mcherry. In conditions with 10 uM IKK2 inhibitor (Millennium), 1 or 5 ug TLR4 antagonist (CRX-526, Glaxo Smith Kline), cells were pre-treated for 1 hr at 37 degrees, 5% CO2. Cells were then treated with 10 ng/ml LPS (Sigma) for 6 hrs. In the conditions with 1.5 ug antagonist, cells were treated with antagonist +/− LPS simultaneously. After 6 hr LPS stimulation, cells were harvested and analyzed for luciferase activity using Steady Glo Reagent (Promega).

### DETAILED DESCRIPTION OF THE INVENTION

Chronic, low-grade inflammation, particularly in adipose tissue, is an important modulator of obesity-induced insulin resistance, although the precise mechanisms initiating this inflammatory response are unclear. The toll-like receptor 4 (Tlr4) is a key initiator of inflammatory responses in macrophages. Given that fatty acids are a bona fide ligand activator of Tlr4 and are also elevated in obesity, and Tlr4 signaling in macrophages plays an important role in obesity-induced inflammation and insulin resistance.

Considering the clear role of macrophages in the propagation of inflammatory signals in adipose tissue and liver (i.e.; through the liver-specific macrophage cell type, the Kupffer cell), it was hypothesized that knockout of Tlr4 signaling in hematopoietic-derived cells (which includes macrophages), would reduce obesity-related increases in macrophage infiltration and inflammation and subsequently prevent in vivo insulin resistance. To address this hypothesis, mice were generated with Tlr4 deleted exclusively in hematopoietic cells. Our results reveal that mice deficient in Tlr4 in their hematopoietic compartment are protected from high-fat diet (HFD) and obesity-induced insulin resistance, in parallel with reduced macrophage infiltration in adipose tissue, reduced chemokine and Iymphokine secretion, and a marked reduction in inflammation in adipose and liver.

To this end, bone marrow transplantation (BMT) was performed on Tlr4lps-del or control C57BI/10J bone marrow cells into irradiated wild type C57BI6 recipient mice to generate hematopoietic cell specific Tlr4 deletion mutant (BMT-Tlr4^{-/-}) and control (BMT-wt) mice, respectively. With this approach, BMT-Tlr4^{-/-} mice have a deficiency of Tlr4 in all hematopoietic cells, including immune cells such as macrophages, but normal Tlr4 expression in all other tissues. When mice were fed a high-fat diet (HFD) for 16 weeks, BMT-wt mice developed obesity, hyperinsulinemia, glucose intolerance and insulin resistance. In contrast, BMT-Tlr4^{-/-} mice became obese, but did not develop fasting hyperinsulinemia, and had an improved response during insulin tolerance tests, compared to HFD BMT-wt mice. The HFD BMT-Tlr4/− mice showed markedly reduced adipose tissue inflammatory markers and macrophage contentcompared to HFD BMT-wt mice. Hyperinsulinemiceuglycemic clamp experiments revealed that hepatic insulin sensitivity after 16 wk HFD, was significantly greater in BMT-Tlr4^{-/-} vs. BMT-wt mice. The suppression of fatty acid concentration during the clamp was also greater in BMT-Tlr4^{-/-} vs. BMT-wt mice, suggesting increased adipose tissue insulin sensitivity. Interestingly, knockdown of Tlr4 in HSC using a lentiviral vector also prevented insulin resistance in HFD mice, further confirming that the loss of Tlr4 in bone marrow derived cells leads to insulin sensitivity. In summary, the results indicate that Tlr4 signaling in hematopoietic-derived cells is important for the development of hepatic and adipose tissue insulin resistance in obese mice.

Knockout of Tlr4 which is a key receptor involved in activation of the innate immune/inflammatory response, in hematopoietic cells, prevents HFD/obesity-induced hyperinsulinemia, hyperglycemia, and abrogates insulin resistance in liver and adipose tissue. Importantly, the improved insulin action in adipose tissue and liver of these mice occurred in conjunction with reduced macrophage infiltration of adipose tissue, as well as reduced expression of proinflammatory cytokines, such as TNF-α, both in adipose tissue and liver.

It was further verified the importance of hematopoietic cell Tlr4 in the induction of insulin resistance by using a gene therapy approach to knockdown Tlr4 in autologous hematopoietic stem cells. Considering Tlr4 is a bona fide receptor for fatty acids, the results suggest that Tlr4 acts as an important transducer of the extracellular signal from fatty acids to activation of intracellular inflammatory pathways in hematopoietic cells (most likely macrophages), with subsequent release of proinflammatory cytokines that cause insulin resistance.

Indeed, several recent studies have demonstrated that mice with knockout of Tlr4, 11, or a loss-of-function mutation in Tlr4, 14, are protected against fatty acid- and obesity-induced insulin resistance. Thus, these studies demonstrate that Tlr4 could play a role in regulating the development of insulin resistance in response to HFD. However, since Tlr4 is expressed in many important insulin-responsive cell types (e.g. muscle, adipocytes, hepatocytes), a limitation of these studies is that they do not specifically isolate the contribution of the innate immune system (e.g. macrophage, neutrophils etc.) to any change in insulin sensitivity.

To address this and other questions, BMT was used to generate chimeric mice with knockout of Tlr4 specifically in hematopoietic cells. Because innate immune cells are derived from hematopoietic stem cells, the model results in the knockout of Tlr4 in macrophages among other hematopoietic cells. Interestingly, the results demonstrate that BMT-Tlr4^{-/-} mice are protected against HFD/obesity-induced hyperinsulinemia, insulin intolerance, and insulin resistance in adipose tissue and the liver. These results are in line with recent studies in which it has been found that myeloid-specific knockdown of JNK1 (which is a downstream target of Tlr4 signaling) and IKK. improve insulin sensitivity in HFD fed mice. Two previous studies have examined the issue of insulin resistance in mice in which Tlr4 is either knocked out or disabled 11,14. In the paper by Shi et al. 11, the authors show that the Tlr4 knockout protects animals from the effects of acute lipid infusions to cause insulin resistance, however, the tissues responsible for this systemic effect could not be specified. This finding during acute lipid infusions did not translate that well into the setting of chronic HFD. Thus, the authors found that the Tlr4 deletion had no effect on body weight or insulin sensitivity in HFD fed male mice, but did lead to increased obesity with insulin sensitivity in females. In contrast, Tsukomo et al. 14 studied mice with a loss of function Tlr4-mutation and found that male animals gained less body weight than controls on HFD, and became less insulin resistant. However, in the setting of a lean and insulin sensitive phenotype, it is not clear whether it is the leanness of the mouse, or the knockout of Tlr4, per se, which causes the insulin sensitivity, and the tissue type responsible for the phenotype could not be determined Since the chimeric mice used herein express Tlr4 deficiency only in bone marrow derived hematopoietic cells, both control and knockout mouse models gained an equal amount of weight on HFD and had equal expansion of both subcutaneous and visceral adipose depots. ThuS, differences in adiposity, between the two groups (BMT-wt and BMT-Tlr4^{-/-}) is not a confounding factor making it possible to ascertain the contribution of hematopoietic cell Tlr4 signaling to insulin sensitivity. It is not clear why the Shi et al., and Tsukomo et al. studies are so different, but perhaps it is due to the fact that one group studied Tlr4 null animals 1\ whereas, the other studied a mouse strain carrying a loss of function mutation in the Tlr4 receptor 14. Of course, other strain differences may also be contributing factors. Clearly, the animal model used herein is much different, adoptive transfer was employed to generate chimeric animals in which case the Tlr4 depletion is only carried in hematopoietic-derived cells with normal Tlr4 in all other tissues.

It is of interest, that while substantial effects were found of the hematopoietic Tlr4 knockout to cause systemic insulin sensitivity, these effects were primarily manifested in liver and adipose tissue. With respect to skeletal muscle, no changes were observed in the insulin stimulated in vivo glucose disposal rate, and since 70-80% of in vivo insulin stimulated glucose disposal is into skeletal muscle, this implies no major changes in skeletal muscle insulin sensitivity. It is well known that on HFD, large increases in macrophage numbers occur in adipose tissue and that, in the liver, Kupffer cell inflammatory activation state is enhanced, and the number of Kupffer cells may also be increased. On the other hand, there are relatively few macrophages that appear in skeletal muscle on RFD, and these cells are mostly present in inter-muscular adipose deposits. Since inflammatory markers were markedly decreased in liver and adipose tissue of the BMT-Tlr4^{-/-} mice, the data indicate that Tlr4 expression in hematopoietic derived cells is an important control point for HFD-induced inflammation in adipose tissue in liver, but much less so in skeletal muscle. In skeletal muscle, it is possible that the Tlr4 on the muscle cell itself plays the major role in detecting lipid signals and in the setting of HFD induced skeletal muscle insulin resistance. Indeed, Tsukomo et al. provide evidence for this hypothesis, since the authors have directly shown that, when studied ex vivo, Tlr4 knockout muscle is protected from fatty acid induced insulin resistance. This is consistent with the results herein which show that deletion of hematopoietic cell Tlr4 is sufficient to cause a systemic insulin sensitive phenotype, but that this was primarily manifested in liver and adipose tissue and not muscle.

Adipose tissue from obese mice and humans is infiltrated with immune cells, particularly bone marrow derived macrophages 2-4,16. The majority of these macrophages surround dying or dead adipocytes 17,18, where they act to clear cellular debris. In order to recruit additional immune cells, these macrophages secrete a wide array of proinflammatory cytokines and chemokines, such as TNF-α and MCP-1. It is believed that these proinflammatory cytokines can induce insulin resistance in nearby cells (e.g. adipocytes), via paracrine effects. Tlr4 in macrophages is necessary for activation of inflammatory pathways by fatty acids or lipopolysaccharides (LPS). Knockdown of Tlr2 and/or Tlr4 in macrophage cells attenuates fatty acid-induced activation of Jnk1 and abrogates TNF-α secretion into the media. In fact, JNK1 is an obligatory component for the ability of fatty acids and Tlr4 to activate inflammatory pathways, and to increase TNF-α secretion 4. In support of these findings, it was found that the expression of several proinflammatory cytokines, namely, IL-6, TNF-α and IL12p7G was markedly reduced in adipose tissue from BMT-Tlr4^{-/-} mice on HFD. It is likely that the reduced expression of TNF-α is directly related to the reduced macrophage infiltration, as macrophages are the primary source of TNF-α in obese adipose tissue 2,3. It is notable that IL-12 p7G was reduced in mice, since IL-12 is important for the transition of naive T-cells into Th1 cells 19. This is relevant to the study herein since the main targets of Th1 cells are macrophages, whereby Th1 cells act to induce a macrophage proinflammatory state. Thus, it was hypothesized that Tlr4 is an obligate receptor for the transduction of an obesity derived signal (Le. increased fatty acids) to macrophages. In turn, activation of inflammation via macrophage Tlr4 potentiates recruitment of additional cells t adipose tissue, with subsequent polarization to a proinflammatory state. This feed-forward process is likely exacerbated by the fact that Tlr4 expression is increased in macrophages in obese adipose tissue 4. Consistent with this, insulin's effect to suppress circulating FFA levels was much greater in the BMT-Tlr4^{-/-} mice, indicative of improved adipose insulin action.

In the liver, macrophages are present in the form of Kupffer cells. Unlike adipose tissue, the content of Kupffer cells does not increase with obesity, but instead they are polarized toward a more proinflammatory state. Because Kupffer cells are bone-marrow derived, our model allows us to determine the effect of Tlr4 in Kupffer cells on induction of inflammation (and insulin resistance) in the liver of obese animals. Similar to results in adipose tissue, it was found that the expression of various proinflammatory markers was markedly reduced in the liver of BMT-Tlr4^{-/-} mice. In fact, the HFD-induced increase in TNF-α and RANTES in liver was completely reversed in BMT-Tlr4^{-/-} mice. In parallel, with this decrease in inflammation, the ability of insulin to suppress HGP in BMT-Tlr4^{-/-} mice was normalized to values seen in chow fed mice. Importantly, these changes occurred despite the fact that liver weight and visceral fat content in HFD fed BMT-Tlr4^{-/-} mice was similar to HFD fed BMT-wt mice. These results suggest that activation of inflammatory pathways in Kupffer cells is necessary for induction of hepatic insulin resistance.

These findings were extended by demonstrating that transplantation of mice with bone marrow containing lentiviral-driven siRNA knockdown of Tlr4 leads to improved insulin sensitivity on HFD. Interestingly, this occurred despite the fact that the results did not achieve as high a level of knockdown as seen in the BMT-Tlr4^{-/-} mice (>95% versus 80% knockdown for BMT-Tlr4^{-/-} and LV-siTlr4, respectively), suggesting that complete knockdown of Tlr4 is not necessary in order for beneficial metabolic effects to occur.

### Definitions

The use of the word “a” or “an” when used in conjunction with the term “comprising” in the claims and/or the specification may mean “one,” but it is also consistent with the meaning of “one or more,” “at least one,” and “one or more than one.”

The term “antagonist” as used herein means a molecule that partially or completely inhibits by any mechanism, an effect of another molecule such as a receptor. As used herein a “Tlr4 antagonist” or a “compound reactive with Tlr4” describes a molecule that is capable of directly or indirectly substantially counteracting, reducing or inhibiting TLr4 biological activity or Tlr4 receptor activation. Such antagonists may be, for example, small organic molecules, peptide chains, antibodies, antibody fragments, MIMETIBODY™ peptide chains or polynucleotides. Such antagonists, may for example, disrupt the activity of TLr4 by preventing activation or formation of functional complexes comprising Tlr4. The Tlr4 antagonist useful in the invention may have the properties of binding a Tlr4 receptor and inhibiting Tlr4 receptor-mediated signaling.

Without limiting the scope the antagonist utilized in the present invention is a lipid A mimetic, CRX-526, by Glaxo Smith Kline. Some examples of other specific Tlr4 antagonists are disclosed in U.S. patent application Ser. No. 12/262,699 the specification of which is incorporated herein in its entirety. The antagonists include, mAb MTS510, antagonists such as Tlr4-ECD which compromises the extracellular domain of a hTLr4A fused to an Fc domain and others. mAb MTS510 is a monoclonal rat antibody of the IgG2a isotype which binds Mus musculus (mouse) Tlr4 and is capable of binding mTlr4 complexed with MD2 as well as inhibiting Tlr4 activity. TLr4-ECD type constructs can also inhibit Tlr4 activity and are believed to antagonize Tlr4 by inhibiting the interaction of MD2 with Tlr4 thus preventing the LPS binding MD2 peptide chain from activating Tlr4. The nucleic acid sequences and polynucleotide compositions disclosed in U.S. patent application publication no. 2009/0142778, the disclosure of which is incorporated by reference in its entirety, has also shown to be effective in eliciting an immune response. The immune response may be a result of the nucleic acid sequence interfering with Tlr4 expression and/or activity.

Tlr4 antagonists useful in the methods of the invention may also be nucleic acid molecules. Such nucleic acid molecules may be interfering nucleic acid molecules such as short interfering RNAs or antisense molecules that are Tlr4 antagonists. Alternatively, polynucleotide molecules such as double or single stranded plasmid DNA vectors, artificial chromosomes, or linear nucleic acids, or other vector that encode a Tlr4 antagonist, or function as a Tlr4 antagonist, may be used in the methods of the invention to administer a Tlr4 antagonist to a subject.

The term “antibodies” is used in meant in a broad sense and includes immunoglobulin or antibody molecules including polyclonal antibodies, monoclonal antibodies including murine, human, humanized, and chimeric monoclonal antibodies and antibody fragments. “Antibody fragments” means a portion of an intact antibody, generally the antigen binding or variable region of the intact antibody. The term “antigen” as used herein means any molecule that has the ability to generate antibodies either directly or indirectly. Included within the definition of antigen is a protein encoding nucleic acid.

In some embodiments of the methods of the invention the TLr4 antagonist is an isolated antibody reactive with Tlr4. An antibody is reactive with a TLr4 when, for example, it specifically binds a given Tlr4 peptide chain or a complex comprising Tlr4. The binding of an antagonist, such as an antibody, reactive with Tlr4, is specific for a given peptide chain when such binding can be used to detect the presence of a first peptide chain, but not a second non homologous peptide chain. This specific binding can be used to distinguish the two peptide chains from each other. Specific binding can be assayed using conventional techniques such as ELISAs and Western Blots as well as other techniques known in the art.

Exemplary antibody antagonists may be abtiboides of the IgG, IgD, IgGa, or IgM isotypes. Additionally, such antagonist antobodies can be post translatioanlly modified by processes such as glcosylation, isomerization, aglycoslation, pegylation, lipidation and the like. Antibody antagonist molecules binding a given Tlr4 homolog with a desired affinity may be selected from libraries of protein variants or fragments by techniques including antibody maturation and other art recognized techniques suitable for non antibody molecules.

The term “subject” means any mammal including humans.

The term “in combination with” as used herein means that the described agents can be administered to a subject together in a mixture, concurrently or as a single agents or sequentially as single agents in any order.

The term “therapeutically effective amount” means those doses that when given to a subject, prevent one or more symptoms of a condition.

The term “preventing” refers to reducing the likelihood that the recipient will incur or develop any of the pathological conditions described herein.

The term “treating” refers to mediating a disease or condition and preventing, or mitigating, its further progression or ameliorate the symptoms associated with the disease or condition.

### Materials and Methods

Methods and materials are described herein. However, methods and materials similar or equivalent to those described herein can be also used to obtain variations of the present invention. The materials, methods, and examples are illustrative only and not intended to be limiting.

### Bone Marrow Transplantation

Murine total bone marrow hematopoietic progenitor donor cells were harvested from wild type or Tlr41ps-del C57B10 mice and available through Jackson Laboratories, Bar Harbor, Me. and transplanted via tail vein injection into lethally irradiated C57B16J mice (1100 rads; Cobalt-60 source) with a minimum cell dose of 106 mononuclear cells, or 100,000 lineage depleted cells per mouse. Transplanted mice were housed in micro-isolator housing for 6 weeks prior to challenge with high fat diet and subsequent insulin sensitivity analyses. For experiments where lentiviral vectors were used to knockdown Tlr4, bone marrow from wild-type CD45.1 mice (back-crossed to C57B16J mice and available through Jackson Laboratories, Bar Harbor, Me.) was lineage depleted for hematopoietic progenitor cell enrichment (as per manufacturers' instruction, Stem Cell Technologies, Vancouver). Transduction of the progenitor and stem cells was performed as described below.

### Lentiviral Vectors:

The third generation lentiviral vectors used in these studies have been described, but in brief, contain the self-inactivating deletion, and an internal CMV promoter driving the marker gene GFP 20. The small-interfering RNA cassette directed against Tlr4 and driven by human H1 pol III promoter, was place upstream of the CMV promoter, cloning details provided upon request. Lentiviral vector supernatants were prepared as previously described 21.

### Transduction

The protocol for efficient transduction of lineage depleted hematopoietic stem cells and progenitors has been previously described 22. Briefly, transduction conditions involved 2 days of prestimulation in serum-free expansion medium (SFEM) with 50 ng/ml of each stem cell factor, thrombopoietin, and flt-3 ligand (Stem Cell Technologies, Vancouver, BC), followed by a high multiplicity of infection transduction of blood cells by pelleting up to 500,000 cells and resuspending approximately 30-100 J . . . 1 L of concentrated virus with a titer greater than 10E9 HeLaTU1 mL and the volume of virus adjusted to ensure a minimum of 100 infectious units per cell . . . Incubation was for 1 hour, followed by addition of 150 f.tL of SFEM medium and cytokines overnight. A second hit was then performed using an additional 30-50 f.tL of high titer virus directly to the cells in medium and incubated an additional night. All incubations are performed at 37″C with 5% CO2. Expansion during 4-day transduction was approximately 2-3 fold.

### Metabolic Studies (ITT and GTT)

Insulin tolerance tests (ITT) were performed pre-and post- diet and following Tlr4 knockdown in all groups of animals. ITT testing allowed the determination of effectiveness of insulin to reduce fasting glucose levels. Briefly, mice were fasted 6 hours, blood glucose concentrations were assessed before the injection of 0.5 U/kg insulin (intraperitoneal injection) and then 10, 15, 20, 30, 45, 60 and 90 min following injection. At each time point a 5 111 blood sample was collected via tail nick and glucose assessed with LifeScan OneTouch@ glucose monitoring system. One week later, the same group of animals were subjected to a glucose tolerance test (GTT) (31). Here, animals were fasted 6 hours, blood glucose concentrations was assessed before and 10, 15, 20,30, 45, 60 and 90 min after the injection of 19/kg 50% dextrose (454 mg/ml).

### Hyperinsulinemic-Euglycemic Clamp

Hyperinsulinemic-euglycemic clamps were conducted to determine insulin stimulated glucose disposal rate (IS-GOR) and the inhibitory effect of insulin on hepatic glucose production (HGP). Briefly, mice were anesthetized with ketamine (80 mg/kg), acepromazine (0.5 mg/kg), and xylazine (16 mg/kg) via IP injection. The jugular vein was cleared of surrounding tissue and two microurethane catheters (Type MRE-025) were advanced −1 cm into the vessel and secured with 4-0 silk suture. The catheters were tunneled to the mid-scapular region and externalized. The skin was closed with 6-0 suture and the catheters were secured within silastic tubing (0.078″ 10×0.125″ 00) that had been externalized and secured to the skin with 6-0 silk suture. The mice were allowed to recover for five days before undergoing the clamp protocol. Following a 6 hour fast, blood glucose was assessed via tail nick, body mass was measured, and the mice were placed in a Lucite restrainer (Braintree Scientific, Braintree, Mass.). Once in the restrainer, −75˜I of whole blood was collected for the assessment of plasma insulin and free fatty acids (at t=−60 min). Equilibrating tracer solution (41.6˜Ci 3H/ml at 2˜1/min) was infused intravenously for 60 min. At the end of the equilibration period (t=0 min), 2×15˜I of whole blood was collected, and blood was deproteinized for the assessment of tracer specific activity and basal glucose disposal rate. Following the equilibration period, a cocktail containing 8% BSA, insulin and tracer was infused at a constant rate (6.0 mU/kg/min and 41.6˜Ci/ml at 2.0˜1/min) along with a variable glucose infusion (50% dextrose, 454 mg/ml). Blood glucose was assessed every 10 min for determination of glucose infusion rate. Glucose infusion rate was adjusted until steady state blood glucose (120 mg/dl, ±5 mg/dl) was achieved. The clamp was terminated when steady state conditions were maintained for ˜30 min (−120 min), at which time 2×15˜I of blood was collected for assessment of tracer specific activity and insulin-stimulated glucose disposal rate (t=−120 min). At the end of the clamp period the mouse were exsanguinated by cardiac puncture (:2:1 ml, whole blood collected) and tissues were harvested, mass recorded and preserved as required for future analysis.

### Tissue Collection and Analysis

At the end of each clamp study, muscle, liver and fat tissues were harvested for measuring of the mRNA and protein content of insulin signaling molecules, and inflammatory signaling molecules. Real Time RT-PCR was performed using ABI systems 9600 thermal cycler, primers sequences available upon request. ELiSAs were performed as per manufacturer's instructions (Simon please provide). MRI analyses including adipose volume determinations were performed using UCSD functional MRI core facility using the 7T system (21 cm bore, Bruker Avance II console). Adipose tissue immunohistochemistry were performed via cryostat sectioning followed by staining with Mac2 antibody (BD Pharmingen) with analysis of crown-like structures as defined previously by Muranoet al. 18

The invention is further described in the following examples, which do not limit the scope of the invention described in the claims.

### EXAMPLES

The present invention is more particularly described in the following examples, which are intended as illustrative only since numerous modifications and variations thereof will be apparent to those of ordinary skill in the art.

### Example 1

### Deletion of Tlr4 Exclusively in Immune Cells Does Not Alter Peripheral Blood Hematopoietic Lineage Distributions

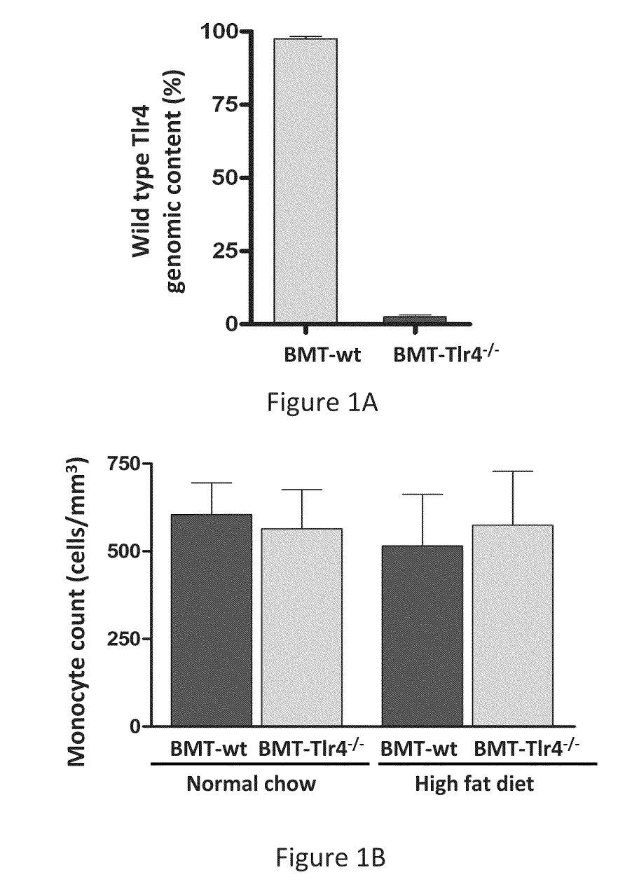

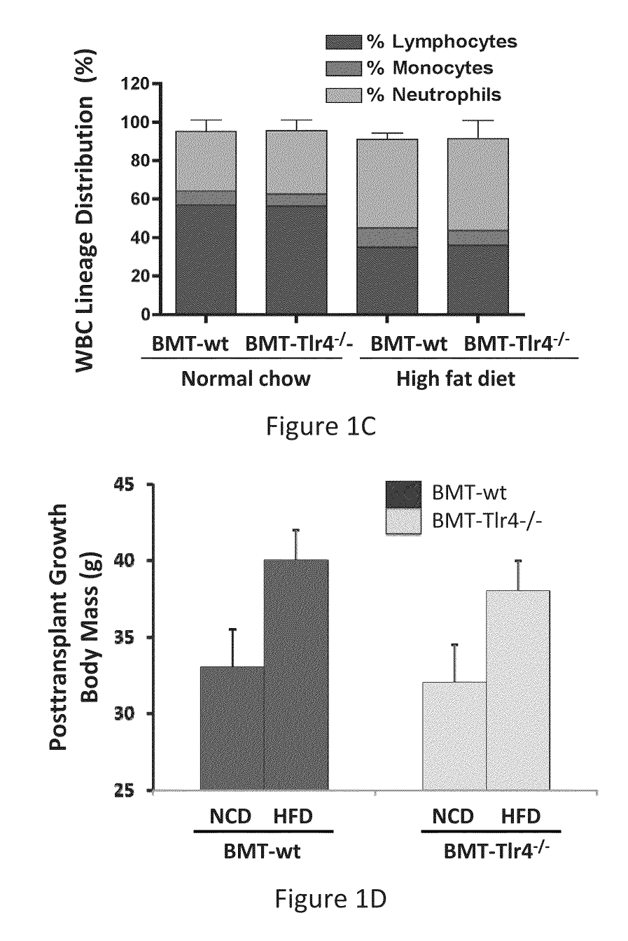

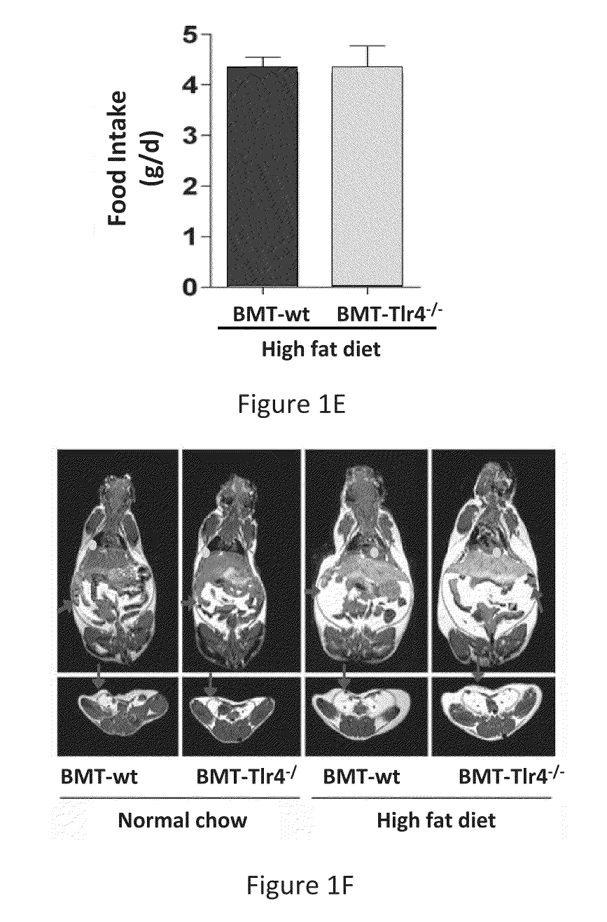

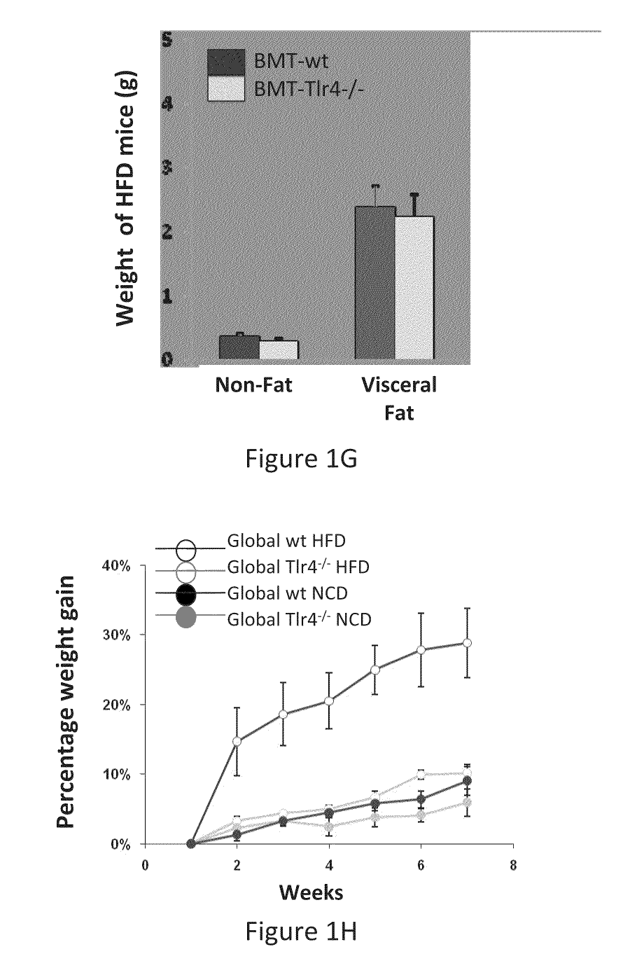

To generate mice with a complete knockout of Tlr4 in macrophages and other immune cells, irradiated wild type (wt) C57BL6 mice were transplanted with bone marrow from Tlr4^{Ips.del 15} or wt C57BL/10J mice. This adoptive transfer approach yielded chimeric mice that were deficient in Tlr4 (BMT-Tlr4^{-/-}) in all hematopoietic derived cells, but which had normal Tlr4 expression in all non-hematopoietic tissues such as skeletal muscle, hepatic tissue, and adipose tissue. Using this technique, eight weeks following bone marrow transplantation (BMT), >95% of white blood cells from BMT-Tlr4^{-/-}mice lacked Tlr4 (FIG. 1A). Mice transplanted with bone marrow from wild type C57BL/10J mice (BMT-wt) displayed normal Tlr4 expression in all hematopoietic derived cells and non-hematopoietic cells/tissues. The loss of Tlr4 did not alter hematopoietic cell lineage distribution, since monocyte, lymphocyte, and neutrophil counts were normal in the BMT-Tlr4^{-/-}mice and BMT-wt mice (FIG. 1B and FIG. 1C), though the proportions of the three cell types differed slightly between normal chow diet (NCO) and high fat diet (HFD) fed mice.

### Example 2

### Hematopoietic Cell Deletion of Tlr4 Does Not Prevent Obesity, But Ameliorates High-Fat-Dietlobesity-Induced Hyperinsulinemia

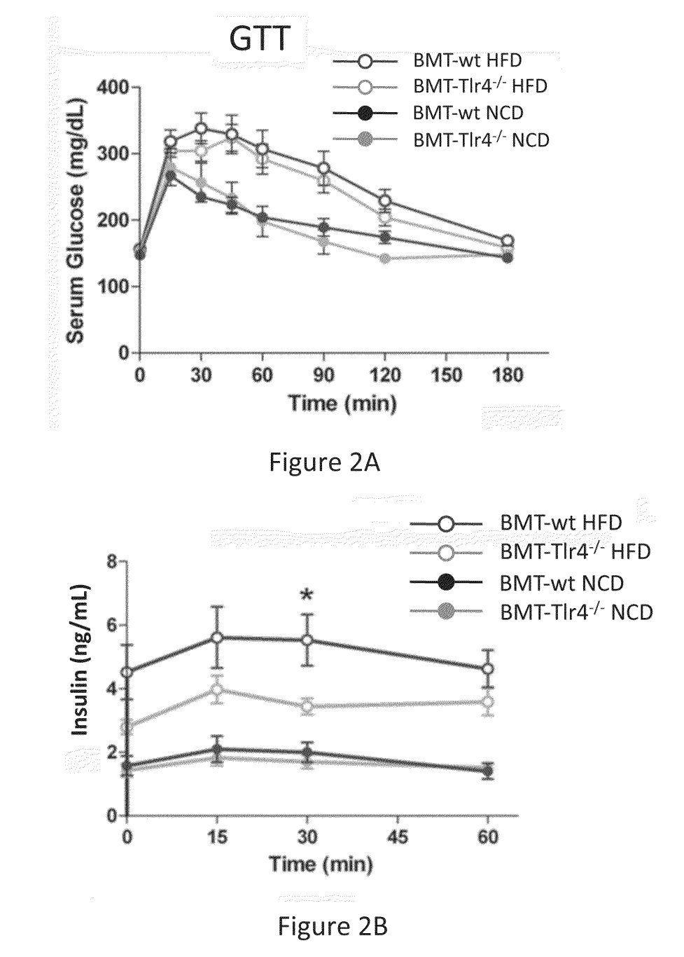

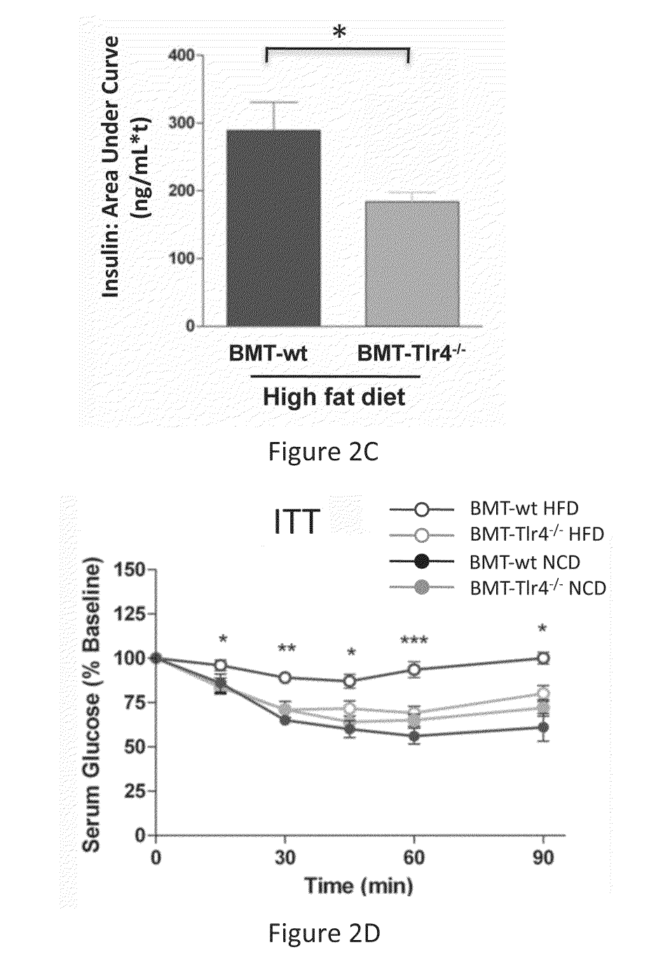

As expected, body weight gain in mice fed a high fat diet (HFD) significantly outpaced mice fed normal chow diet (NCD). There were no significant body weight differences between BMT-wt or BMT-Tlr4^{-/-}mice and no differences in food intake were detected (FIG. 1D and FIG. 1E). In vivo volumetric analysis of body composition using magnetic resonance imaging (MRI) revealed a marked increase in liver size with severe hepatic steatosis, as well as increased visceral adipose deposition in HFD versus NCD mice, irrespective of BMT donor type cells (FIG. 1F). These results demonstrate that the loss of Tlr4 did not affect the ability of mice to become obese; moreover, it did not affect the distribution of fat in the obese animal (FIG. 1G). In contrast, on HFD, body weight gain was markedly reduced in the global Tlr4 knockout mice (i.e. non transplanted mice with knockout of Tlr4 in all tissues) compared to wt controls (FIG. 1H) comparable to the results of Tsukomo et al. (15). The glucose response during a glucose tolerance test (GTT) was increased in HFD fed mice, and there were no statistical differences between the BMT-wt and BMT-Tlr4^{-/-} mice (FIG. 2A). On HFD, mice become hyperinsulinemic, but the insulin response during the GTT was significantly lower in HFD BMT-T1r4^{-/-} versus BMT-wt mice (FIG. 2B and FIG. 2C), indicating an overall improvement in insulin action as a result of Tlr4 deletion from hematopoietic cells. Next an insulin tolerance test (ITT) was performed, which shows that while HFD BMT-T1r4^{-/-} caused insulin resistance in BMT-wt mice, the hypoglycemic response in the HFD group was comparable to NCD fed mice (FIG. 2D), directly demonstrating protection from insulin resistance in these mice. Thus, the deleterious effects of HFD/obesity on glucose metabolism and insulin sensitivity were significantly improved in HFD BMT-Tlr4^{-/-} mice, suggesting that Tlr4 in immune cells plays an important role in mediating the effects of obesity on insulin action.

### Example 3

### Hematopoietic Cell Specific Deletion of Tlr4 Improves Insulin Sensitivity in Liver and Adipose Tissue

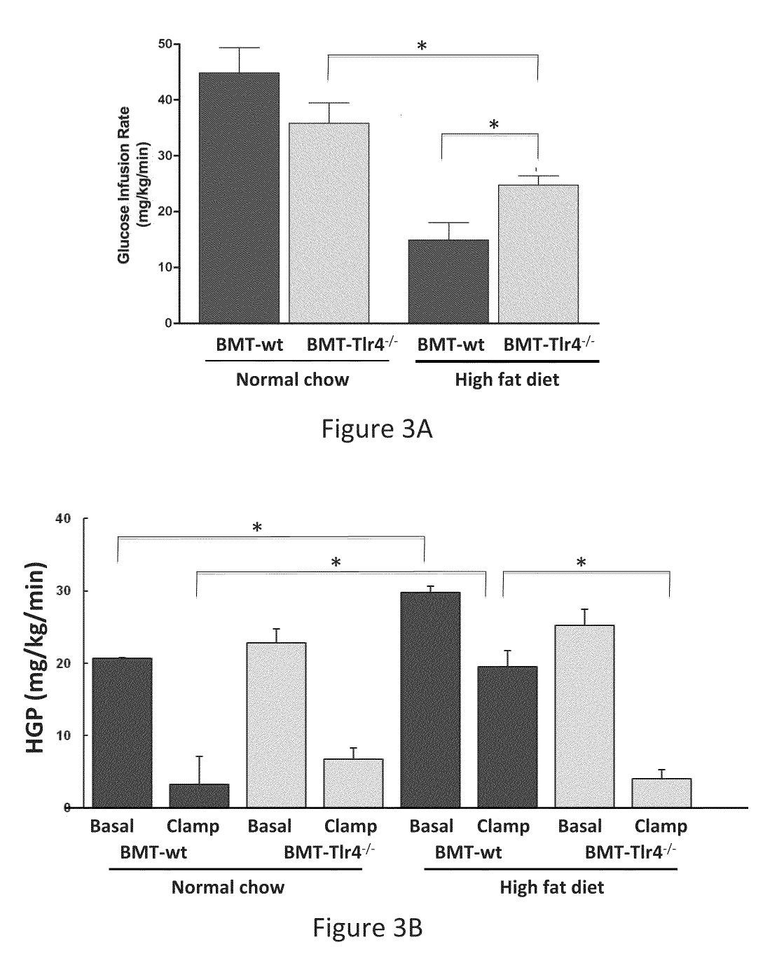

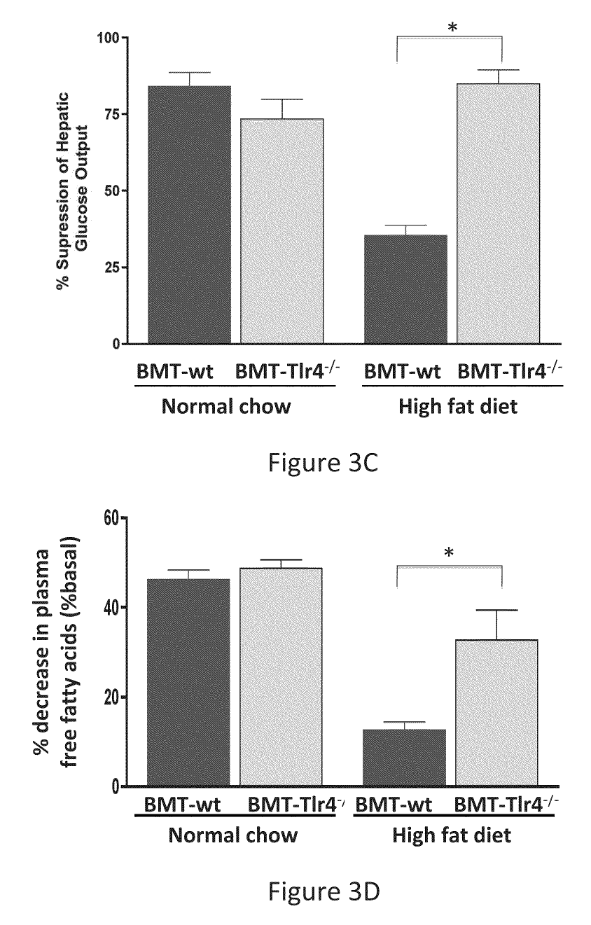

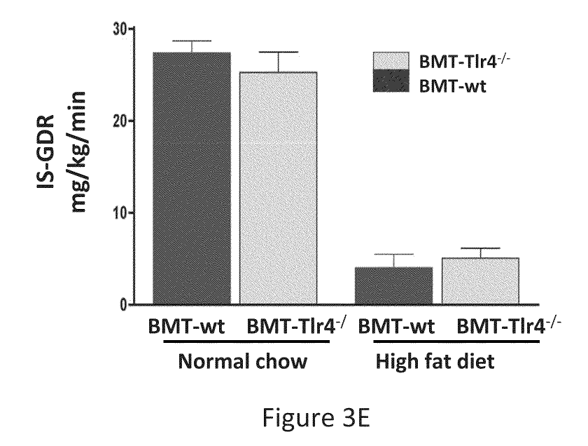

To further quantify whole-body insulin sensitivity and to better delineate the tissue-specific site(s) responsible for the improved glucose homeostasis in BMT-Tlr4^{-/-} mice, hyperinsulinemic-euglycemic clamp studies were performed. With this procedure the measuring of glucose infusion (GINF) rate was required to keep a constant level of blood glucose during a simultaneous infusion of insulin, and the higher the GINF, the greater the overall insulin sensitivity. The clamp results showed that the GINF required to maintain euglycemia (−125 mg/dL) was not significantly different between NCD fed BMT-wt and BMT-Tlr4^{-/-} mice. As expected, BMT-wt mice fed HFD had markedly decreased GINF values, confirming insulin resistance. In contrast, GINF values were ˜70% higher in the HFD BMT-Tlr4^{-/-} mice compared to wt (FIG. 3A) demonstrating partial protection from HFD-induced insulin resistance. During the clamp studies, the steady-state insulin and glucose concentrations were the same between groups. To determine the contribution of hepatic glucose production, a simultaneous infusion of tracer labeled glucose was infused during the clamp study to provide a measure of the rate of glucose disposal (Rd). Subtracting the GINF from the total Rd yields the endogenous glucose production rate (mainly from liver). As compared to NCO mice, HFD BMT-wt mice, displayed increased basal hepatic glucose production (HGP) with a markedly impaired ability of insulin to suppress HGP during the clamp (FIG. 3B and FIG. 3C), demonstrating hepatic insulin resistance. In contrast, in HFD BMT-Tlr4^{-/-} mice, basal HGP and insulin suppression of HGP was completely normalized to values seen in NCO mice (FIG. 3B and FIG. 3C). Adipose tissue insulin sensitivity was assessed by measuring the percent decrease in plasma free fatty-acid concentration during the clamp study, and was also normalized in the HFD BMT-Tlr4^{-/-} mice (FIG. 3D). Notably, the HFD induced impairment in insulin-stimulated glucose disposal rate (IS-GDR) in BMT-wt, which primarily reflects skeletal muscle insulin sensitivity, was not prevented in BMT-Tlr4^{-/-} mice (FIG. 3E). Taken together, these data demonstrate that Tlr4 deficiency in hematopoietic cells prevents HFD-induced glucose intolerance and insulin resistance, primarily via effects in the liver and adipose tissue.

### Example 4

### Inflammatory Cytokine Signaling Is Mediated by Macrophages in Obese HFD BMT-Tlr4-1-mice

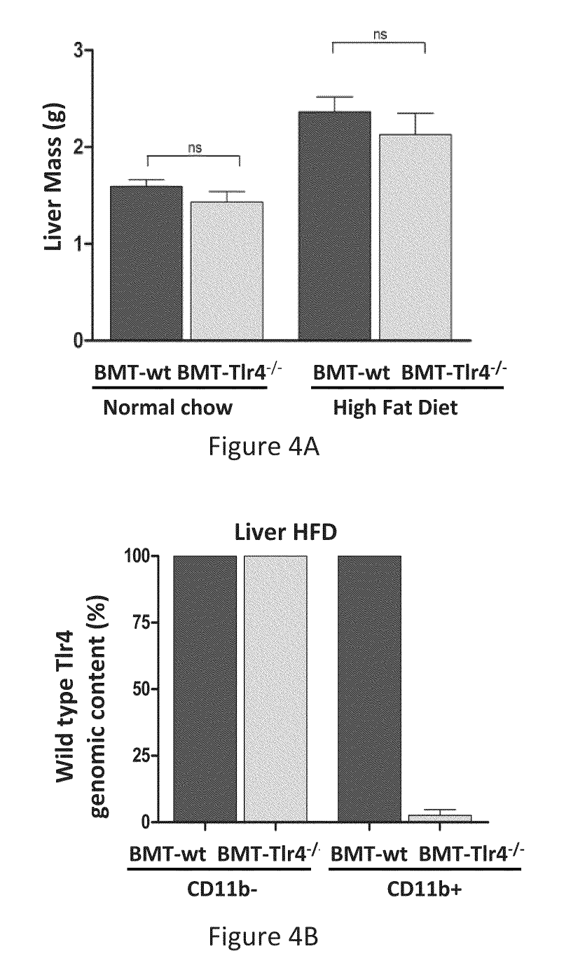

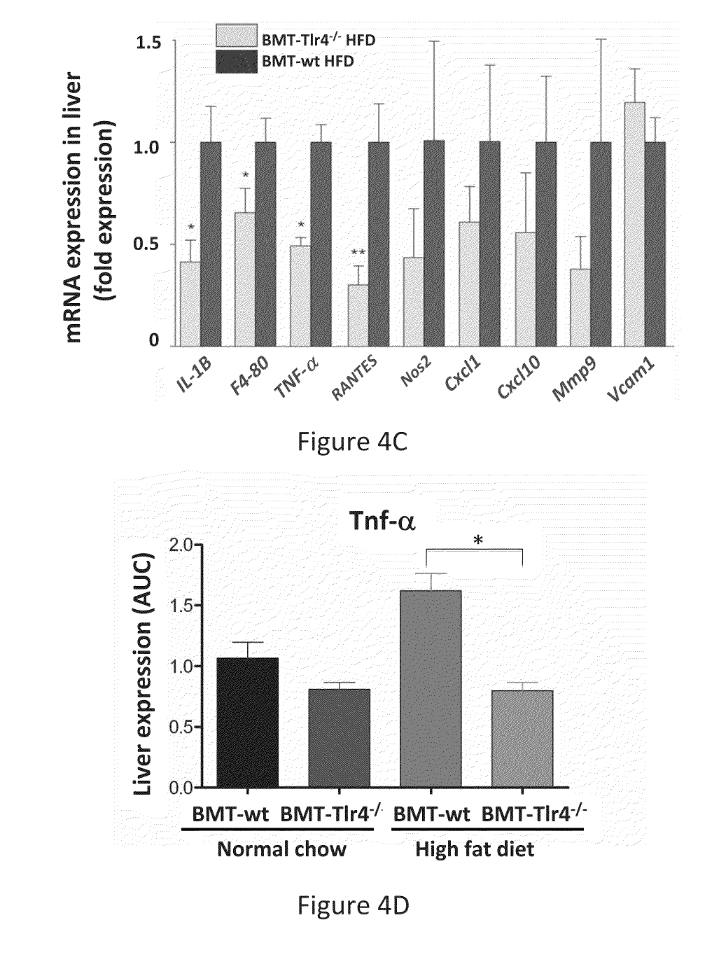

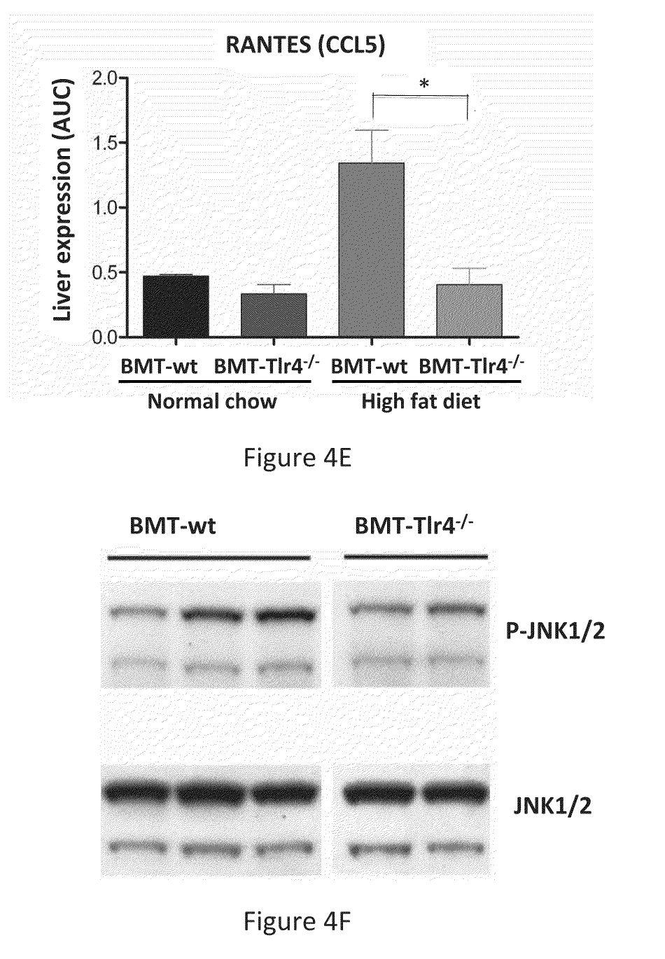

Given that macrophages are the somatic cell with the highest surface expression of Tlr4, and, as such, are potent sensors and effectors for Tlr4 mediated inflammatory responses, various inflammatory markers were assessed in liver, adipose tissue and skeletal muscle. Consistent with the MRI data in FIG. 1F, HFD led to an increase in liver weights (FIG. 4A). To demonstrate that the Kupffer cell population in the recipient mice had been replaced by the transplanted hematopoietic cells, cells were isolated from the livers of HFD BMT mice and subjected them to flow cytometry sorting. Using CD11b as a Kupffer cell marker, it was found that cells lacking CD11b had normal Tlr4 content, whereas the Kupffer cells (CD11b positive) were almost completely devoid of Tlr4 in HFD mice (FIG. 4B). This demonstrates that the transplanted Tlr4^{-/-} hematopoietic cells fully reconstitute the Kupffer cell population in these mice. In liver tissues, mRNA expression analyses of macrophage-specific immune activators, such as IL-1β and F4/80 were significantly reduced by 60% and 30%, respectively, in HFD BMT-Tlr4^{-/-} versus HFD BMT-wt mice (FIG. 4C). Similarly, levels of other macrophage expressed inflammatory genes, such as TNF-α, and RANTES (Cel5) were also significantly reduced (50% and 70%, respectively). Moreover, immune regulators such as Nos2, Cxcl1, Cxcl10 and Mmp9 tended towards reduced expression. As a control, the endothelial cell specific marker VCAM1 showed no statistically significant change in level of expression between BMT-wt and BMT-Tlr4^{-/-} mice. Complementing the changes in gene expression, the protein content in liver tissue of TNF-α, and RANTES was also significantly reduced (FIG. 4D and FIG. 4E). Western blot analysis of phosphorylated c-Jun N-terminal kinase 1 and 2 (JNK1/2) was also reduced in the livers of HFD fed BMT-Tlr4^{-/-} versus BMT-wt mice (FIG. 4F), which is consistent with the known involvement of JNK1/2 signaling in macrophages and inflammation. Taken together, these results indicate that the reduction in liver inflammatory signaling in the BMT-Tlr4^{-/-} mice results from decreased inflammatory signals in the liver-specific macrophage cell type, Kupffer cells.

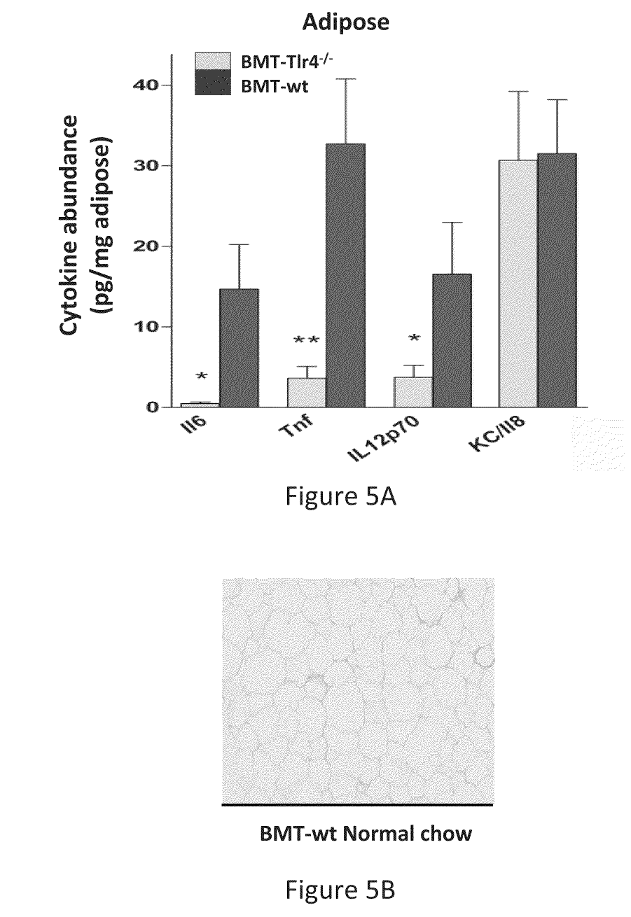

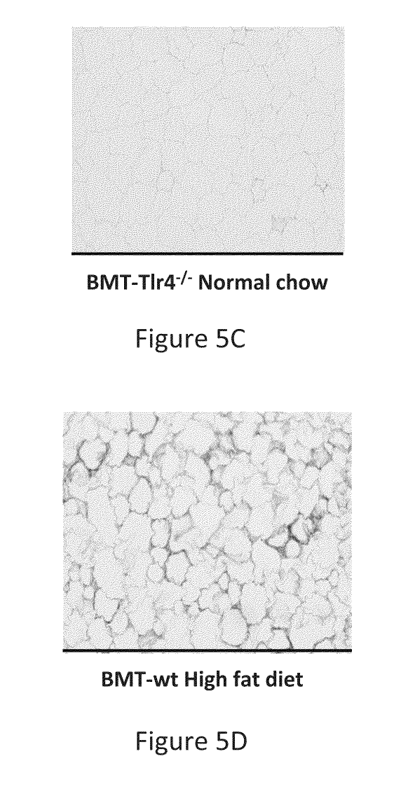

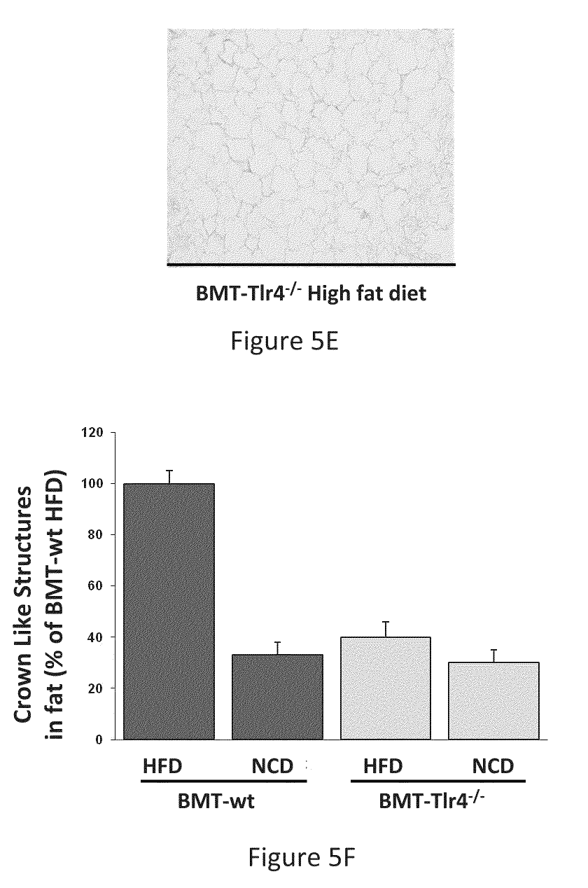

In adipose tissue, the protein amounts of TNF-α, IL-6 and IL-12p70 were also significantly reduced (FIG. 5A). Because macrophages are an important source of TNF-α and IL-6 in adipose tissue 2,3, macrophage infiltration was measured in adipose tissue from HFD BMT-wt and BMT-Tlr4^{-/-} mice. Staining of adipose tissue from HFD BMT-wt mice demonstrated gross infiltration of macrophages using macrophage specific antibody MAC2, as compared to NCD mice (FIG. 5D, compare to panels B and C). Interestingly, this infiltration was completely prevented in BMT-Tlr4^{-/-} mice (FIG. 5E), as measured by the number of crown-like structures (macrophages) present in the extracellular space between adipocytes (FIG. 5F). These findings are consistent with the aforementioned reductions in gene expression and protein content of proinflammatory signaling molecules in both liver and adipose tissue.

### Example 5

### Knockdown of Tlr4 Using Lentiviral Vector Gene Transfer in Hematopoietic Cells Causes Improved Insulin Sensitivity in HFD Mice

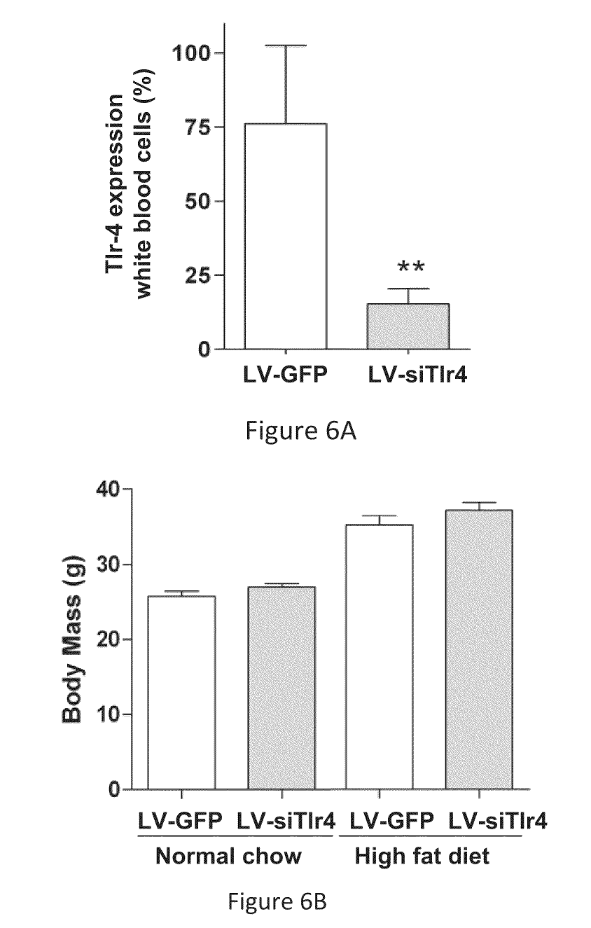

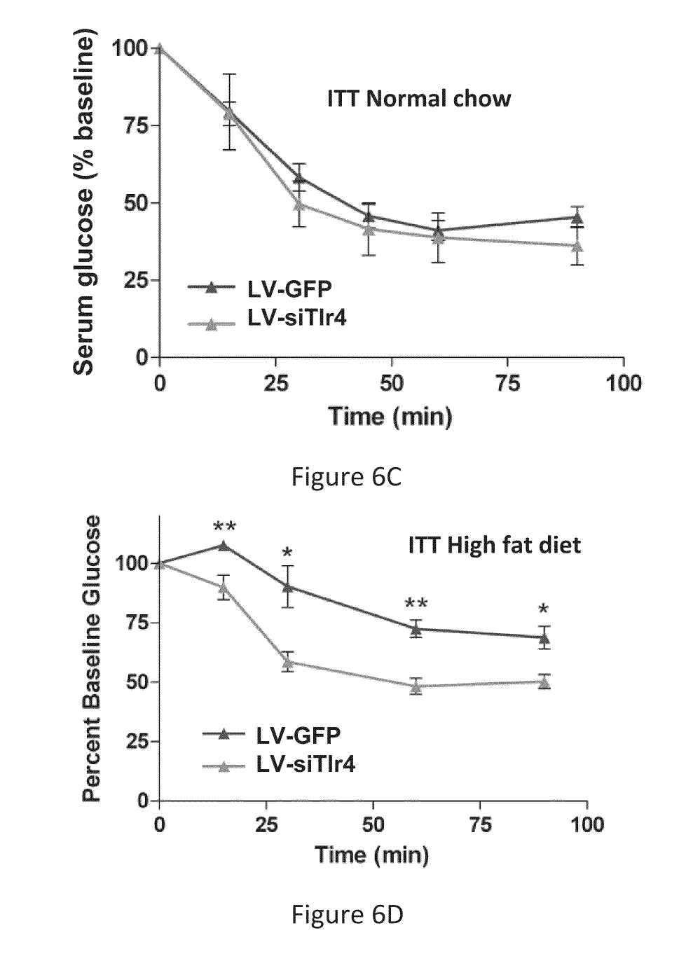

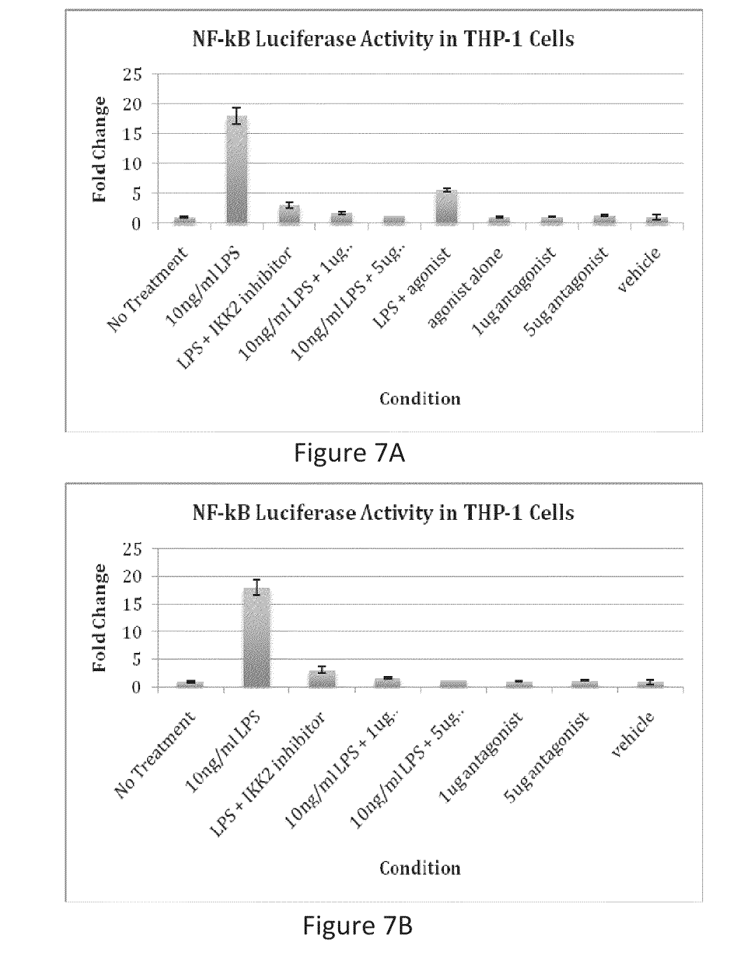

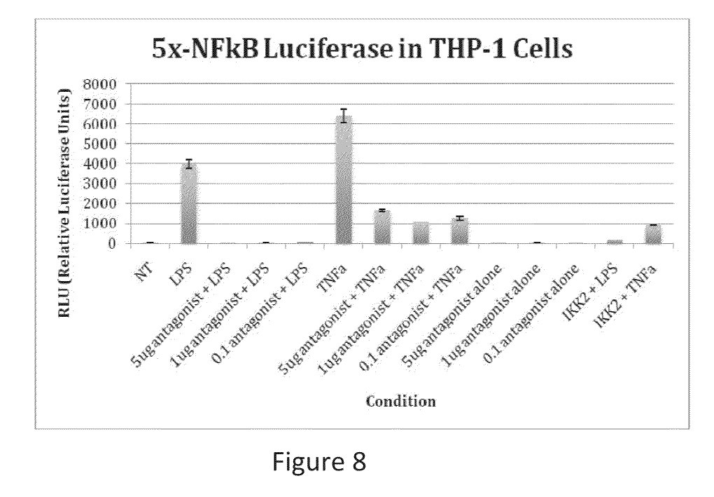

To verify the importance of hematopoietic Tlr4 in mediating obesity-induced insulin resistance in mice, a novel approach was employed using lentiviral vectors to knockdown Tlr4 in autologous hematopoietic stem cells. Bone marrow hematopoietic and progenitor cells from wild-type C57B16 donor mice were transduced in vitro with lentiviral vectors expressing a small interfering RNA (siRNA) targeted against endogenous Tlr4 (LV-siTlr4) or a control vector. To ensure high-level transduction efficiency in the bone marrow cells, the marker gene GFP was included in both vectors. Transduced cells were then transplanted into irradiated C57B16 recipients and after 8 weeks for bone marrow reconstitution, bone marrow cells from these primary BMT mice were then sorted by flow cytometry for GFP expression. Using this approach, the GFP-positive cells represent the bone marrow cell population that was successfully and stably transduced with the LV-siTlr4 or control vector. These GFP-positive bone marrow cells were then transplanted into irradiated C57B16, secondary recipient mice. Endogenous levels of Tlr4 in peripheral blood cells was knocked down by ˜80% in LV-siTlr4 mice, as compared to control LV-GFP mice (FIG. 6A). As expected on HFO both transplanted mouse groups gained a comparable amount of body weight (FIG. 6B). On normal chow diet, both the LV-siTlr4 mice and the control vector mice displayed insulin sensitivity during ITTs (FIG. 6C). When the same mice were placed on HFO, the LVshTlr4 mice retained normal insulin sensitivity, while the control vector mice became insulin resistant (FIG. 6D). These results confirm the role of hematopoietic Tlr4 expression in obesity-induced insulin resistance, and raise the possibility of a gene therapy approach for treatment of obesity-induced insulin resistance.

### Methods of Screening Test Compounds

Also included in the present invention are methods for screening test compounds, or rather potential Tlr4 antagonists, e.g. lipids, polypeptides, polynucleotides, inorganic or organic large or small molecule test compounds, to identify antagonists useful in the treatment of disorders associated with insulin resistance. The methods include using rational drug design methods to identify test compounds that interfere with Tlr4 expression or activity. Various screening assays may be employed to determine if the test compounds modulate Tlr4 expression or activity. For example, coupling one of MD-2, LPS, or TLr4 with a label, e.g., a radioisotope or non-isotopic label, such that binding of MD-2 to LPS or Tlr4 can be determined by detecting the labeled compound in a complex. Alternatively, LPS or Tlr4 may be coupled with a radioisotope or enzymatic label to monitor the ability of a test compound to modulate binding to LPS in a complex. Compounds that interfere with Tlr4 expression and/or activity can also be identified using cell based or cell free assays, as are known in the art.

### Pharmaceutical Compositions

Antagonists useful in the methods of the invention may be prepared in a pharmaceutical composition containing an effective amount of the antagonist as an active ingredient. It is envisioned that, for administration to a host, TLR ligands, other cell surface ligands, cytokines or growth factors, soluble TLRs, antibodies, other inhibitory factors, and stimulated/differentiated cells will be suspended in a formulation suitable for administration to a host. Aqueous compositions of the present invention comprise an effective amount of ligand, factor or cells dispersed in a pharmaceutically acceptable formulation and/or aqueous medium. The phrases “pharmaceutically and/or pharmacologically acceptable” refer to compositions that do not produce an adverse, allergic and/or other untoward reaction when administered to an animal, and specifically to humans, as appropriate.

As used herein, “pharmaceutically acceptable carrier” includes any solvents, dispersion media, coatings, antibacterial and/or antifungal agents, isotonic and/or absorption delaying agents and the like. The use of such media or agents for pharmaceutical active substances is well known in the art. Except insofar as any conventional media or agent is incompatible with the active ingredient, its use in the therapeutic compositions is contemplated. Supplementary active ingredients can also be incorporated into the compositions. For administration to humans, preparations should meet sterility, pyrogenicity, general safety and/or purity standards as required by FDA Office of Biologics standards. In addition to FDA standards, Remington: The Science and Practice of Pharmacy, 21^{st} Edition is herein incorporated by reference.

Soluble receptors, antibodies, inhibitory factors or cells, ligands, or cells for administration will generally be formulated for parenteral administration, e.g., formulated for injection via the intravenous, intramuscular, sub-cutaneous, intralesional, or even intraperitoneal routes. The preparation of an aqueous composition that contains cells as a viable component or ingredient will be known to those of skill in the art in light of the present disclosure. In all cases the form should be sterile and must be fluid to the extent that easy syringability exists and that viability of the cells is maintained. It is generally contemplated that the majority of culture media will be removed from cells prior to administration.

Generally, dispersions are prepared by incorporating the various soluble receptors, antibodies, inhibitory factors, or viable cells into a sterile vehicle which contains the basic dispersion medium and the required other ingredients for maintaining cell viability as well as potentially additional components to effect proliferation or differentiation in vivo. Upon formulation, solutions will be administered in a manner compatible with the dosage formulation or in such amount as is therapeutically effective. Some variation in dosage will necessarily occur depending on the condition of the subject being treated. The person responsible for administration will, in any event, determine the appropriate dose for the individual subject.

In some embodiments, the methods include preventive methods or a method of treatment. e.g. administering a therapeutically effective amount of a composition described herein to a subject who is at risk of having insulin resistance and obesity, e.g. subjects at the highest risk for developing type 2 diabetes.

Dosage, toxicity, and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site affected tissue to minimize the potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods described herein, the therapeutically effective dose can be estimated initially from cell culture assays. A dose may be formulated in animal models to achieve a circulating plasma concentration of the test compound as determined in cell culture. Such information can be used to more accurately determine useful doses in humans. Levels in plasma may be measured, for example, by high performance liquid chromatography.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or the age of the subject, and other diseases present. Moreover, treatment of a subject with a therapeutically effective amount of the compositions described herein can include a single treatment or a series of treatments.

### Administration

Administration to a human is most preferred. The human to whom the compounds and compositions of the present invention are administered has a disease or condition in which control blood glucose levels are not adequately controlled without medical intervention, but wherein there is endogenous insulin present in the human's blood. Non-insulin dependent diabetes mellitus (NIDDM) is a chronic disease or condition characterized by the presence of insulin in the blood, even at levels above normal, but resistance or lack of sensitivity to insulin action at the tissues. The compounds and compositions of the present invention are also useful to treat acute or transient disorders in insulin sensitivity, such as sometimes occur following surgery, trauma, myocardial infarction, and the like. The compounds and compositions of the present invention are also useful for lowering serum triglyceride levels. Elevated triglyceride level, whether caused by genetic predisposition or by a high fat diet, is a risk factor for the development of heart disease, stroke, and circulatory system disorders and diseases. The physician of ordinary skill will know how to identify humans who will benefit from administration of the compounds and compositions of the present invention.

The compositions are formulated and administered in the same general manner as detailed herein. The compounds of the instant invention may be used effectively alone or in combination with one or more additional active agents depending on the desired target therapy. Furthermore, it will be understood by those skilled in the art that the compounds of the present invention, including pharmaceutical compositions and formulations containing these compounds, can be used in a wide variety of combination therapies to treat the conditions and diseases described above. The present invention can be used in combination with modulators, such as fibrates, in the treatment of cardiovascular disease, and in combination with PPARγ modulators, such thiazolidinediones, in the treatment of diabetes, including non-insulin dependent diabetes mellitus and insulin dependent diabetes mellitus, and with agents used to treat obesity) and with other therapies, including, without limitation, chemotherapeutic agents such as cytostatic and cytotoxic agents, immunological modifiers such as interferons, interleukins, growth hormones and other cytokines, hormone therapies, surgery and radiation therapy.

The development of suitable dosing and treatment regimens for using the particular compositions described herein in a variety of treatment regimens, including e.g. oral, parenteral, intravenous, intranasal and intramuscular administration and formulation, is well known in the art, some of which are briefly described below for the purpose of illustration.

In certain applications the pharmaceutical compositions described herein may be delivered via oral administration to a subject. As such, these compositions may be formulated with an inert diluent or with an assimilable edible carrier, or they may be enclosed in hard or soft shell gelatin capsule, or they may be compressed into tablets, or they may be incorporated directly with food of the diet. The active compounds may even be incorporated with excipients and used in the form of ingetable tablets, buccal tablets, troches, capsules, elixirs, suspensions, syrups, wafers, and the like. Other oral administrations may include the compound incorporated with one or more excipients in the form of mouthwash, dentrifrice, buccal tablet, oral spray, or sublingual orally-administered formulation.

In certain circumstances it will be desirable to deliver the pharmaceutical compositions disclosed herein parenterally, intravenously, intramuscularly, or even intraperitoneally. The pharmaceutical compositions may alternatively delivered by intranasal sprays, inhalation, and/or aerosol delivery vehicles.

In certain embodiments, liposomes, nanocapsules, microparticels, lipid particles, vesicles and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nonoparticle or the like. Alternatively, compositions of the present invention can be bound, either covalently or non-covalently, to the surface of such vehicle carriers. The preparation of these potential drug carriers is generally known to those of skill in the art.

### Incorporation by Reference

Throughout this application, various publications, patents, and/or patent applications are referenced in order to more fully describe the state of the art to which this invention pertains. The disclosures of these publications, patents, and/or patent applications are herein incorporated by reference in their entireties, and for the subject matter for which they are specifically referenced in the same or a prior sentence, to the same extent as if each independent publication, patent, and/or patent application was specifically and individually indicated to be incorporated by reference.

### Other Embodiments

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Facchini, F. S., Hua, N., Abbasi, F. & Reaven, G. M. Insulin resistance as a predictor of age-related diseases. J Clin Endocrinol Metab 86, 3574-3578 (2001).
2. Weisberg, S. P., et al. Obesity is associated with macrophage accumulation in adipose tissue. The Journal of clinical investigation 112, 1796-1808 (2003).
3. Xu, H., et al. Chronic inflammation in fat plays a crucial role in the development of obesity-related insulin resistance. The Journal of clinical investigation 112, 18211830 (2003).
4. Nguyen, M. T., et al. A subpopulation of macrophages infiltrates hypertrophic adipose tissue and is activated by FFAS via TLR2, TLR4 and JNK-dependent pathways. The Journal of biological chemistry (2007).
5. Arkan, M. C., et al. IKK-beta iinks inflammation to obesity-induced insulin resistance. Nat Med 11,191-198 (2005).
6. Solinas, G., et al. JNK1 in hematopoietically derived cells contributes to dietinduced inflammation and insulin resistance without affecting obesity. Cell Metab 6, 386-397 (2007).
7. Aderem, A. & Ulevitch, R. J. Toll-like receptors in the induction of the innate immune response. Nature 406, 782-787 (2000).
8. Wolowczuk, I., et al. Feeding our immune system: impact on metabolism. Clin Dev Immuno/2008, 639803 (2008).
9. Lee, J. Y., Sohn, K. H., Rhee, S. H. & Hwang, D. Saturated fatty acids, but not unsaturated fatty acids, induce the expression of cyclooxygenase-2 mediated through Toll-like receptor 4. The Journal of biological chemistry 276, 16683-16689 (2001).
10. Lee, J. Y., et al. Reciprocal modulation of Toll-like receptor-4 signaling pathways involving MyD88 and phosphatidylinositol 3-kinase/AKT by saturated and polyunsaturated fatty acids. The Journal of biological chemistry 278, 37041-37051 (2003).
11. Shi, H., et al. TLR4 links innate immunity and fatty acid-induced insulin resistance. The Journal of clinical investigation 116, 3015-3025 (2006).
12. Horowitz, J. F., Braudy, R. J., Martin, W. H., 3rd & Klein, S. Endurance exercise training does not alter lipolytic or adipose tissue blood flow sensitivity to epinephrine. Am J Physio/277, E325-331 (1999).
13. Jensen, M. D., Haymond, M. W., Rizza, R. A., Cryer, P. E. & Miles, J. M. Influence of body fat distribution on free fatty acid metabolism in obesity. The Journal of clinical investigation 83, 1168-1173 (1989).
14. Tsukumo, D. M., et al. Loss-of-function mutation in Toll-like receptor 4 prevents diet-induced obesity and insulin resistance. Diabetes 56, 1986-1998 (2007).
15. Poltorak, A., et al. Defective LPS signaling in C3H/HeJ and C57BL110ScCr mice: mutations in Tlr4 gene. Science (New York, N.Y 282, 2085-2088 (1998).
16. Lumeng, C. N., Deyoung, S. M., Bodzin, J. L. & Saltiel, A. R. Increased inflammatory properties of adipose tissue macrophages recruited during diet-induced obesity. Diabetes 56, 16-23 (2007).
17. Cinti, S., et al. Adipocyte death defines macrophage localization and function in adipose tissue of obese mice and humans. J Lipid Res 46, 2347-2355 (2005).
18. Murano, I., et al. Dead adipocytes, detected as crown-like structures, are prevalent in visceral fat depots of genetically obese mice. J Lipid Res 49, 1562-1568 (2008).
19. Hsieh, C. S., et al. Development of TH1 CD4+ T cells through IL-12 produced by Listeria-induced macrophages. Science (New York, N.Y. 260, 547-549 (1993).
20. Dull, T., et al. A third-generation lentivirus vector with a conditional packaging system. Journal of virology 72, 8463-8471 (1998).
21. Miyoshi, H., Blomer, U., Takahashi, M., Gage, F. H. & Verma, I. M. Development of a self-inactivating lentivirus vector. Journal of virology 72, 8150-8157 (1998).
22. Woods, N. B., Bottero, V., Schmidt, M., von Kalle, C. & Verma, I. M. Gene therapy:therapeutic gene causing lymphoma. Nature 440, 1123 (2006).
23. Production and purification of lentiviral vectors. Tiscornia G, Singer O, Verma I. M. Nat. Protoc. 2006;1(1):241-5.

What is claimed:

1. A method of treating or preventing a disorder associated with insulin resistance in a subject, the method comprising administering to the subject a therapeutically effective amount of a Tlr4 antagonist.

2. The method of claim 1, wherein the disorder associated with insulin resistance is selected from the group consisting of hyperinsulinemia, cardiovascular disease, obesity, and diabetes.

3. A method of reducing inflammatory signaling in a subject, the method comprising administering to the subject a therapeutically effective amount of a Tlr4 antagonist.

4. The method of claim 3, wherein the reducing inflammatory signaling is present in liver tissue.

5. The method of claim 4, wherein the reducing inflammatory signaling in liver tissue is a reduction in IL-1β and/or F4/80 gene expression.

6. The method of claim 4, wherein the reducing inflammatory signaling in liver tissue is a reduction in Nos2, Cxcl1, Cxcl10 and/or Mmp9 gene expression.

7. The method of claim 4, wherein the reducing inflammatory signaling present in liver tissue is a reduction in TNF-α and/or RANTES protein content.

8. The method of claim 3 wherein the reducing inflammatory signaling is present in adipose tissue.

9. The method of claim 8, wherein the reducing inflammatory signaling in adipose tissue is a reduction in TNF-α, IL-6 and/or IL12p70 protein content.

10. The method of claim 1, wherein the Tlr4 antagonist is a monoclonal antibody.

11. The method of claim 10, wherein the monoclonal antibody is an antibody to Tlr4.

12. The method of claim 2, wherein the Tlr4 antagonist is a monoclonal antibody.

13. The method of claim 12, wherein the monoclonal antibody is an antibody to Tlr4.

14. A method of diagnosing a subject with insulin resistance, the method comprising measuring the levels of Tlr4 expression and/or activity in a sample from a subject, wherein an elevated level of Tlr4 expression and/or activity as compared to a reference indicates that the subject has insulin resistance.

15. The method of claim 14, wherein the sample comprises a biological fluid.

16. A method of selecting a Tlr4 antagonist, the method comprising screening for a compound which modulates Tlr4 expression and/or activity.

17. A pharmaceutical composition to treat or prevent a disorder associated with insulin resistance comprising a Tlr4 antagonist and a physiologically acceptable carrier.

18. The pharmaceutical composition of claim 17 wherein the disorder associated with insulin resistance is selected from the group consisting of hyperinsulinemia, cardiovascular disease, obesity, and diabetes.

19. The pharmaceutical composition of claim 17 wherein the Tlr4 antagonist is a lipid A mimetic, CRX-526 and a physiologically acceptable carrier.

20. The method of claim 1, wherein the Tlr4 antagonist is a lipid A mimetic, CRX-526.
